(19) United States
(12) Patent Application Publication
(10) Pub. No.: US 2018/0245123 A1
(43) Pub. Date: Aug. 30, 2018

(54) METHOD AND APPARATUS FOR DETECTING TOTAL COLIFORM AND E. COLI IN POTABLE WATER

(71) Applicants: Sushanta Kumar MITRA, Mississauga, Ontario (CA); Naga Siva Kumar GUNDA, North York, Ontario (CA); Ravi Shankar CHAVALI VENKATA SUBRAMANYA, Edmonton, Albert (CA)

(72) Inventors: Sushanta Kumar MITRA, Mississauga, Ontario (CA); Naga Siva Kumar GUNDA, North York, Ontario (CA); Ravi Shankar CHAVALI VENKATA SUBRAMANYA, Edmonton, Albert (CA)

(21) Appl. No.: 15/752,880

(22) PCT Filed: Aug. 12, 2016
(86) PCT No.: PCT/CA2016/000208

§ 371 Date: Feb. 14, 2018

Related U.S. Application Data
(60) Provisional application No. 62/233,734, filed on Sep. 28, 2015.

(30) Foreign Application Priority Data
Aug. 14, 2015 (IN) 3096/MUM/2015
Jun. 30, 2016 (IN) 201621022603

(51) Int. Cl. C12Q 1/10; C12Q 1/04; G01N 21/78; C12Q 1/40; B01L 3/00

(52) U.S. Cl. CPC C12Q 1/10; C12Q 1/045; G01N 21/78; C12Q 1/40; B01L 2300/045; B01L 3/502; B01L 2300/0832; B01L 2300/0681; B01L 2300/069; C12Y 302/01023

(57) ABSTRACT

An apparatus for testing for the presence of pathogens in test liquids is described. Also described is a method for testing for the presence of pathogens in test liquids. Also described is a hydrogel for testing for the presence of pathogens in test liquids.

### CROSS REFERENCE TO RELATED APPLICATIONS

This application claims priority to Indian patent application no. 3096/MUM/2015 filed on Aug. 14, 2015, U.S. provisional patent application No. 62/233,734 filed on Sep. 28, 2015, and Indian provisional patent application no. 201621022603 filed on Jun. 30, 2016, the entire contents of each of which are incorporated by reference herein.

### FIELD OF THE INVENTION

The present invention relates generally to the field of detecting pathogens in liquid samples, and, more particularly, to a method and apparatus for testing for total coliform and E. coli in potable water.

### BACKGROUND OF THE INVENTION

Access to safe and clean drinking water is becoming a global challenge, particularly for water scarce regions of the world. The most common sources of water regularly used for household and recreational purposes are rivers, lakes, ponds, ground water and wells. These sources are often contaminated with sewage sludge, agricultural wastes, fertilizers, chemicals, organic and inorganic materials, and dead animals in many parts of the world. Such contamination leads to the growth of several water-borne pathogens such as bacteria (e.g., Salmonella, Campylobacter, Shigella, Listeria monocytogenes, Enterobacter and various strains of Escherichia Coli (E. coli)), viruses (e.g., poliovirus, coronaviruses, rotavirus, norwalkvirus and hepatitis), protozoa (e.g., Cryptosporidium), and other parasites (e.g., Helminths) that have significant implications on public health.¹⁻³

Hence, the quality of water consumed from these sources needs to be regularly monitored in order to prevent water-borne diseases. Water quality testing in developing countries and in most of the developed nations currently involves transportation of the water samples to centralized laboratories where qualified personnel conduct laboratory based testing and assessment of water quality.⁴⁻⁶

However, such an approach is time-consuming, requires well-equipped laboratories and availability of adequately trained personnel, which may not be feasible in minimally resourced communities. Hence, extensive testing of water quality on a regular basis would require simple, rapid and field deployable tests that can be performed at the point of use by any unskilled individual.

One of the most common and best approaches to assess the extent of microbial pollution in water is to estimate the density of certain indicator bacteria (e.g., coliforms, fecal coliforms and E. coli) in the water samples to be tested.¹⁻³

The presence of coliform bacteria in water samples is an indicator of the presence of water-borne pathogens and precautionary measures are to be taken in these cases to avoid any outbreaks. In recent years, there have been several approaches that were pursued towards the development of methods for the detection and quantification of total coliform and E. coli in water. The conventional approach for the detection and enumeration of total coliform and E. coli involves filtering the water samples through a membrane filter, followed by counting the number of E. coli colonies of the filtrate sample using plate counting method.⁷⁻¹⁰

The counted colonies can be related to the number of cells based on which the quality of water is determined. However, these methods take 24 to 48 hours to produce results, often requiring transportation of water samples to a central laboratory and trained personnel to perform the test. Alternatively, several rapid detection methods have also been developed using advanced techniques such as quantum dots,¹¹ flow cytometry,¹² polymerase chain reaction (PCR),¹³⁻¹⁵ DNA microarrays,¹⁶⁻¹⁹ enzyme linked immunosorbent assay (ELISA),²⁰ and Fluorescent In-Situ Hybridization (FISH).²¹ However, these methods require the use of sophisticated equipment to perform the tests thereby necessitating the establishment of well-equipped laboratories, which may not be feasible in poorly resourced communities.

The focus of the present invention is on the development of low-cost and rapid methods for the detection of total coliform and E. coli in potable water, which is particularly relevant for limited resource communities and developing economies. One of the most promising approaches to achieve the same, is to identify certain biomarkers (e.g., enzymes) secreted by coliform bacteria using a specific chromogenic or fluorogenic substrate.²²⁻²⁴

The resulting colour intensity or fluorescence intensity from the enzyme-substrate interaction can be correlated to the bacterial concentration in the water sample. The detection of enzymes using defined substrate technique is well known, robust and cost effective. The most commonly used marker enzymes for total coliform and E. coli are β-D-galactosidase (GAL) and β-D-glucuronidase (GUD), respectively. There are several substrates available in the literature for the detection of these enzymes, the most prominent ones being 6-Chloro-β-indolyl-β-D-galactoside (Red-Gal—a chromogenic substrate) to target GAL and 4-Methylumbelliferyl β-D-glucoronide trihydrate (MUG—a fluorogenic substrate) to target GUD.^{23,25,26}

The MUG molecule contains two components: 4-Methylumbelliferyl (4-MU) and β-D-glucuronic acid. Red-Gal contains two components: 6-Chloro-3-indolyl and β-D-galactose. The GAL enzyme produced by coliform bacteria hydrolyses this complex Red-Gal molecule resulting in the release of red colour producing dimerized 6-Chloro-3-indolyl compound.^{23,25,26} The GUD enzyme produced by E. coli hydrolyses this complex MUG molecule resulting in the release of the fluorogenic compound 4-MU. There are several examples of such enzyme-based detection techniques available in the literature. Though these enzyme-based techniques are quite robust, complexities arise in simplifying these test methods for field use, since there are several regulatory issues that need to be addressed in contrast to a laboratory-based method. For example, the United States Environmental Protection Agency (USEPA) requires that the volume of water sample that needs to be tested should be 100 mL since bacteria are not uniformly distributed in water and the bacterial concentrations are reported as CFU/100 mL of contaminated water sample. Hence, most of these tests also include a pre-filtration step in order to concentrate the bacteria from 100 mL of contaminated water samples, which increases the number of steps in performing a field test.

An example of the pre-filtration step from other field tests involves using a syringe to collect and pass 100 mL of test water through filters with a pore size sufficient to trap the bacteria to be tested. Chemical reagents would then be added sequentially to the filters, and subsequent colour changes, or lack thereof, could be used to assess the presence, or absence, of total coliform and E. coli in the water sample.²⁷

This pre-concentration step and the sequential addition of reagents add a certain level of complexity to the field test procedure.

### SUMMARY OF THE INVENTION

One aspect of the invention provides a hydrogel impregnated with one or more substrates for marker enzymes of one or more pathogens for use in a liquid testing kit, wherein when the hydrogel comes into contact with a liquid sample, if the one or more pathogens is present in the liquid sample, the hydrogel and liquid sample change colour due to a reaction process between the one or more substrates and the one or more pathogens.

A further aspect of the invention provides a hydrogel, wherein the one or more pathogens is total coliform.

A further aspect of the invention provides a hydrogel, wherein the one or more pathogens is E. coli.

A further aspect of the invention provides a hydrogel, wherein the one or more pathogens are total coliform and E. coli.

A further aspect of the invention provides a hydrogel, wherein the one or more substrates is 6-Chloro-3-indolyl-β-D-galactoside (Red-Gal).

A further aspect of the invention provides a hydrogel, wherein the one or substrates is 4-Methylumbelliferyl β-D-glucoronide trihydrate (MUG).

A further aspect of the invention provides a hydrogel, wherein the one or more substrates are 6-Chloro-3-indolyl-β-D-galactoside (Red-Gal) and 4-Methylumbelliferyl β-D-glucoronide trihydrate (MUG).

A further aspect of the invention provides a hydrogel, wherein the one or more substrates is 5-bromo-6-chloro-3-indolyl-β-Dgalactopyranoside (Magenta Gal).

A further aspect of the invention provides a hydrogel, wherein the hydrogel also comprises other compounds to accelerate the reaction process.

A further aspect of the invention provides a hydrogel, wherein the other compounds comprise a lysing agent.

A further aspect of the invention provides a hydrogel, wherein the lysing agent is bacteria protein extraction reagent (B-PER).

A further aspect of the invention provides a hydrogel, wherein the other compounds comprise a growth medium.

A further aspect of the invention provides a hydrogel, wherein the growth medium is lauryl tryptose broth (LTB).

A further aspect of the invention provides a dried hydrogel as the hydrogel referred to in the paragraphs above.

Another aspect of the invention provides a hydrogel impregnated with one or more substrates for marker enzymes of one or more pathogens for use in a liquid testing kit, wherein when the hydrogel comes into contact with a liquid sample, if the one or more pathogens is present in the liquid sample, the hydrogel and liquid sample fluoresce under an ultraviolet (UV) lamp due to a reaction process between the one or more substrates and the one or more pathogens.

A further aspect of the invention provides a hydrogel, wherein the one or more pathogens is E. coli.

A further aspect of the invention provides a hydrogel, wherein the one or more substrates is 4-methylumbelliferyl-β-Dgalactopyranoside (MUGal).

A still further aspect of the invention provides an apparatus for testing for the presence of one or more pathogens in a liquid sample, the apparatus comprising: (a) a collection chamber; (b) a plunger comprising at least one plunger wall and a plunger tip, wherein the at least one plunger wall is sized to fit in sliding contact with and move longitudinally within the collection chamber when the plunger is raised or lowered relative to the collection chamber; (c) said plunger tip comprising a filter with pores, said pores sized to permit liquid to pass therethrough, but to not permit the one or more pathogens to pass therethrough; and (d) a testing composition comprising one or more substrates for marker enzymes of one or more pathogens.

A further aspect of the invention provides a testing apparatus, wherein the one or more pathogens is total coliform.

A further aspect of the invention provides a testing apparatus, wherein the one or more pathogens is E. coli.

A further aspect of the invention provides a testing apparatus, wherein the one or more pathogens are total coliform and E. coli.

A further aspect of the invention provides a testing apparatus, wherein the one or more substrates is 6-Chloro-3-indolyl-β-D-galactoside (Red-Gal).

A further aspect of the invention provides a testing apparatus, wherein the one or more substrates is 4-Methylumbelliferyl β-D-glucoronide trihydrate (MUG).

A further aspect of the invention provides a testing apparatus, wherein the one or more substrates are 6-Chloro-3-indolyl-β-D-galactoside (Red-Gal) and 4-Methylumbelliferyl D-glucoronide trihydrate (MUG).

A further aspect of the invention provides a testing apparatus, wherein the one or more substrates is 5-bromo-6-chloro-3-indolyl-β-Dgalactopyranoside (Magenta Gal).

A further aspect of the invention provides a testing apparatus, wherein the testing composition is a hydrogel impregnated with one or more substrates for marker enzymes of one or more pathogens, wherein when the hydrogel comes into contact with the liquid sample, if the one or more pathogens is present in the liquid sample, the hydrogel and liquid change colour due to a reaction process between the one or more substrates and the one or more pathogens.

A further aspect of the invention provides a testing apparatus, wherein the one or more substrates is 4-methylumbelliferyl-β-Dgalactopyranoside (MUGal).

A further aspect of the invention provides a testing apparatus, wherein the testing composition is a hydrogel impregnated with one or more substrates for marker enzymes of one or more pathogens, wherein when the hydrogel comes into contact with the liquid sample, if the one or more pathogens is present in the liquid sample, the hydrogel and liquid change fluoresce under an ultraviolet (UV) lamp due to a reaction process between the one or more substrates and the one or more pathogens.

A further aspect of the invention provides that the hydrogel of the testing apparatus described in the paragraphs above is a dried hydrogel.

A still further aspect of the invention provides a method of testing for the presence of one or more pathogens in a liquid sample, the method comprising: (a) collecting the liquid sample in a collection chamber; (b) inserting into the collection chamber a plunger comprising at least one plunger wall and a plunger tip, wherein the at least one plunger wall is sized to fit in sliding contact with and move longitudinally within the collection chamber when the plunger is raised or lowered relative to the collection chamber, the plunger tip comprising a filter with pores, said pores sized to permit liquid to pass therethrough, but to not permit said one or more pathogens to pass therethrough; (c) moving the plunger tip into the collection chamber such that a quantity of the one or more pathogens is retained on the filter as the liquid is passed therethrough which brings the quantity of one or more pathogens into contact with a testing composition comprising one or more substrates for marker enzymes of the one or more pathogens; and (d) observing the testing composition and liquid for a colour change, said colour change signifying the presence of said one or more pathogens.

A further aspect of the invention provides a testing method, wherein the one or more pathogens is total coliform.

A further aspect of the invention provides a testing method, wherein the one or more pathogens is E. coli.

A further aspect of the invention provides a testing method, wherein the one or more substrates are total coliform and E. coli.

A further aspect of the invention provides a testing method, wherein the one or more substrates is 6-Chloro-3-indolyl-β-D-galactoside (Red-Gal).

A further aspect of the invention provides a testing method, wherein the one or more substrates is 4-Methylumbelliferyl β-D-glucoronide trihydrate (MUG).

A further aspect of the invention provides a testing method, wherein the one or more substrates are 6-Chloro-3-indolyl-β-D-galactoside (Red-Gal) and 4-Methylumbelliferyl β-D-glucoronide trihydrate (MUG).

A further aspect of the invention provides a testing method, wherein the one or more substrates is 5-bromo-6-chloro-3-indolyl-β-Dgalactopyranoside (Magenta Gal).

A further aspect of the invention provides a testing method, wherein the testing composition is a hydrogel impregnated with one or more substrates for marker enzymes of one or more pathogens, wherein when the hydrogel comes into contact with the liquid sample, if the one or more pathogens is present in the liquid sample, the hydrogel and liquid change colour due to a reaction process between the one or more substrates and the one or more pathogens.

A further aspect of the invention provides for a dried hydrogel to be used in the testing methods described in the above paragraphs.

A further aspect of the invention provides that, in the testing methods described above, the colour change is assessed on a quantitative basis by an optical reader, and the results are transmitted to a communications network.

Yet a further aspect of the invention provides a method of testing for the presence of one or more pathogens in a liquid sample, the method comprising: (a) collecting the liquid sample in a collection chamber; (b) inserting into the collection chamber a plunger comprising at least one plunger wall and a plunger tip, wherein the at least one plunger wall is sized to fit in sliding contact with and move longitudinally within the collection chamber when the plunger is raised or lowered relative to the collection chamber, the plunger tip comprising a filter with pores, said pores sized to permit liquid to pass therethrough, but to not permit said one or more pathogens to pass therethrough; (c) moving the plunger tip into the collection chamber such that a quantity of the one or more pathogens is retained on the filter as the liquid is passed therethrough which brings the quantity of one or more pathogens into contact with a testing composition comprising one or more substrates for marker enzymes of the one or more pathogens; and (d) observing the testing composition and liquid for a fluorescence change, said fluorescence change signifying the presence of said one or more pathogens.

A further aspect of the invention provides a testing method, wherein the one or more pathogens is E. coli.

A further aspect of the invention provides a testing method, wherein the one or more substrates is 4-methylumbelliferyl-β-Dgalactopyranoside (MUGal).

A further aspect of the invention provides a testing method, wherein observing the testing composition and liquid for a fluorescence change comprises observing the testing composition and liquid under an ultraviolet (UV) lamp.

### BRIEF DESCRIPTION OF THE DRAWINGS

The foregoing and other aspects of the invention will become more apparent from the following description of specific embodiments thereof and the accompanying drawings which illustrate, by way of example only, the principles of the invention. In the drawings:

FIG. 1 shows an apparatus according to an embodiment of the invention with the plunger out of the collection chamber and the screw cap removed;

FIG. 2 shows the apparatus of FIG. 1 with the plunger partially inserted into the collection chamber;

FIG. 3 shows the apparatus of FIG. 1 with the plunger more fully inserted into the collection chamber, and shading of a test liquid indicating a positive test;

FIG. 4 is a schematic diagram depicting a detection methodology using an apparatus according to an embodiment of the invention;

FIG. 5A is a schematic diagram depicting a variation of the detection methodology of FIG. 4 according to another embodiment of the invention;

FIG. 5B is an illustrative diagram depicting the steps of the detection methodology of FIG. 5A;

FIG. 6 shows embodiments of the apparatus of the invention containing a control sample and containing a test sample, the apparatus on the right containing the test sample and showing development of colour due to the enzymatic reaction of GAL with Red-Gal after 1 hour of incubation at 37° C.;

FIG. 7 shows development of color in samples with decreasing concentrations of E. coli in an embodiment of the apparatus of the invention after 1 hour of incubation at 37° C.;

FIG. 8 shows comparative development of color in a number of test samples after reacting with different enzymatic substrates; and

FIG. 9 shows comparative development of fluorescence in a number of test samples after reacting with different enzymatic substrates.

### DETAILED DESCRIPTION OF EMBODIMENTS

The description which follows, and the embodiments described therein, are provided by way of illustration of examples of particular embodiments of the principles of the present invention. These examples are provided for the purposes of explanation, and not limitation, of those principles and of the invention.

In order to simplify field test methods, the present invention discloses a novel hydrogel-based pathogen detection system, which can simultaneously pre-concentrate the pathogen from water samples and also provide qualitative colorimetric results in a simple three step procedure, and which does not require a separate pre-concentration step, nor the sequential addition of chemical reagents.

The present method described here allows the user to identify total coliform by visualizing the change in colour within minutes of testing contaminated water. In addition, the ease of the testing method makes it a potential candidate for field deployment. The method proposed by the present invention does not require any sophisticated equipment or trained personnel. Furthermore, the present invention does not require temperature regulation or a colour detection module, which has been required by some other similar prior systems. This method has a great potential to be developed into a comprehensive field test kit to be used at the point of source, which would adequately address the needs of the limited resource communities. This method does not require temperature regulation or a colour detection module when seeking qualitative results. Temperature regulation may cause results to be obtained more quickly, in some circumstances. Also, a colour detection module may be necessary when seeking quantitative results.

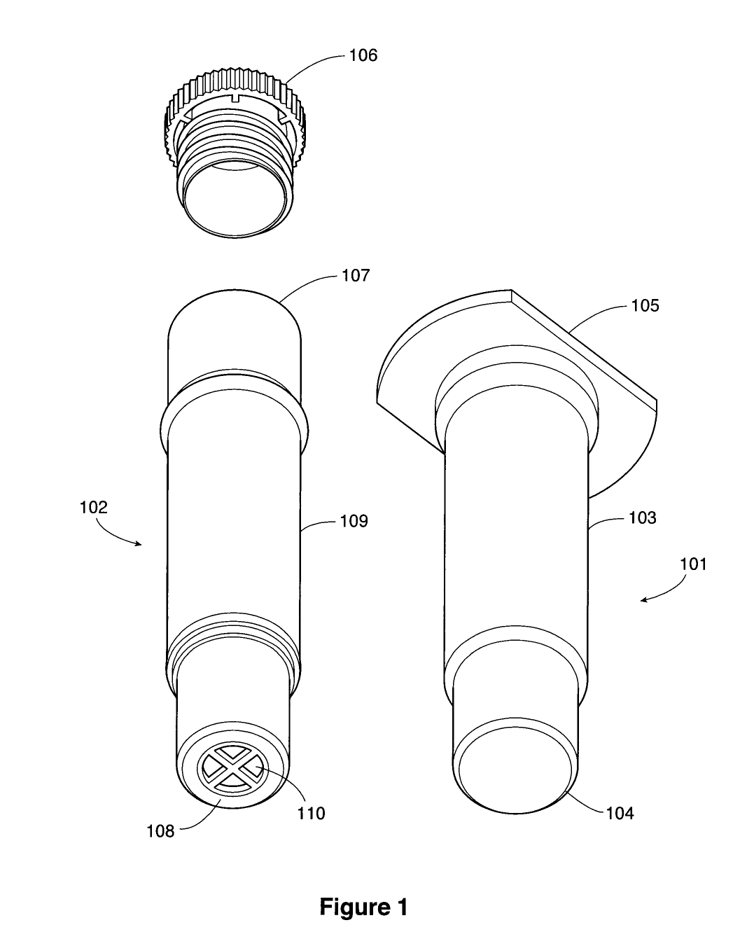

A testing apparatus according to an embodiment of the invention is shown in FIG. 1. As shown in FIG. 1, the testing apparatus comprises a collection chamber 101, and a plunger 102. The collection chamber 101, also referred to as a collection tube, comprises sides 103, a bottom end 104, and a top end 105. The collection chamber 101 is adapted to be able to contain at least 100 mL of water, or other liquid, to be tested. Given that the testing method involves using visual cues to reach conclusions, a transparent or translucent collection chamber is preferred; however, it will be appreciated that an opaque collection chamber could also be used. As such, the collection chamber 101 may be made of plastic or glass, but it will be appreciated that other materials for the collection chamber may be used.

The bottom end of the plunger 102 is a plunger tip 108, and the plunger has plunger wall 109. The plunger 102 may be made of plastic or glass, but it will be appreciated that other materials for the plunger may be used. The plunger 102 may optionally be fitted with a screw top 106 which can be inserted into the top end 107 of the plunger 102. In FIG. 1, the screw top 106 is shown removed from the plunger 102. In some embodiments, when the screw top 106 is inserted into the plunger 102, the screw top 106 may form an airtight seal with the top end 107 of the plunger 102, and in other embodiments the screw top 106 may allow air to escape. In other embodiments, other types of tops, such as snap-fit tops, may be substituted for screw top 106.

The collection chamber 101 is open at the top end 105, but otherwise does not have any openings through which liquid could escape. The collection chamber 101 is generally cylindrical in shape, and the plunger 102 and plunger tip 108 are sized so that they may be freely inserted into the collection chamber 101, and so that the plunger wall 109 is in sliding contact with the inner wall of the sides 103 of the collection chamber 101 when the plunger 102 is moved up and down therein. It will be appreciated that, while various different shapes could be used for the collection chamber 101 and the plunger 102, the plunger should be generally complimentary in shape and size with the collection chamber, and that the plunger tip is generally complimentary in size and shape with the size and shape of the horizontal cross-section of the collection chamber to allow the plunger tip to move up and down within the collection chamber whilst the plunger wall maintains sliding contact against the inner wall thereof.

In some embodiments, such as the embodiment shown in FIG. 1, the plunger tip 108 is fitted with a filter 110 comprising pores. The filter's pores are sized to permit water or other liquids to pass through it, but to conversely not allow certain pathogens to pass through. It will be appreciated that by varying the size of the pores, different pathogens will be able to pass through the filter and/or be trapped by the filter, thus allowing for the presence of different pathogens to be tested.

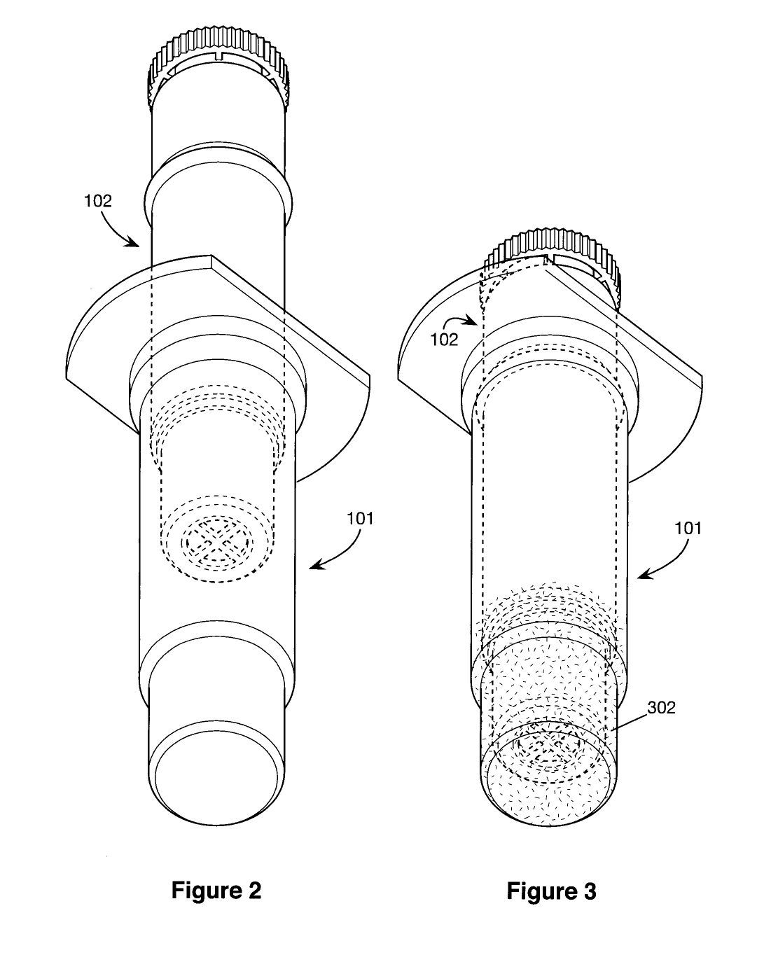

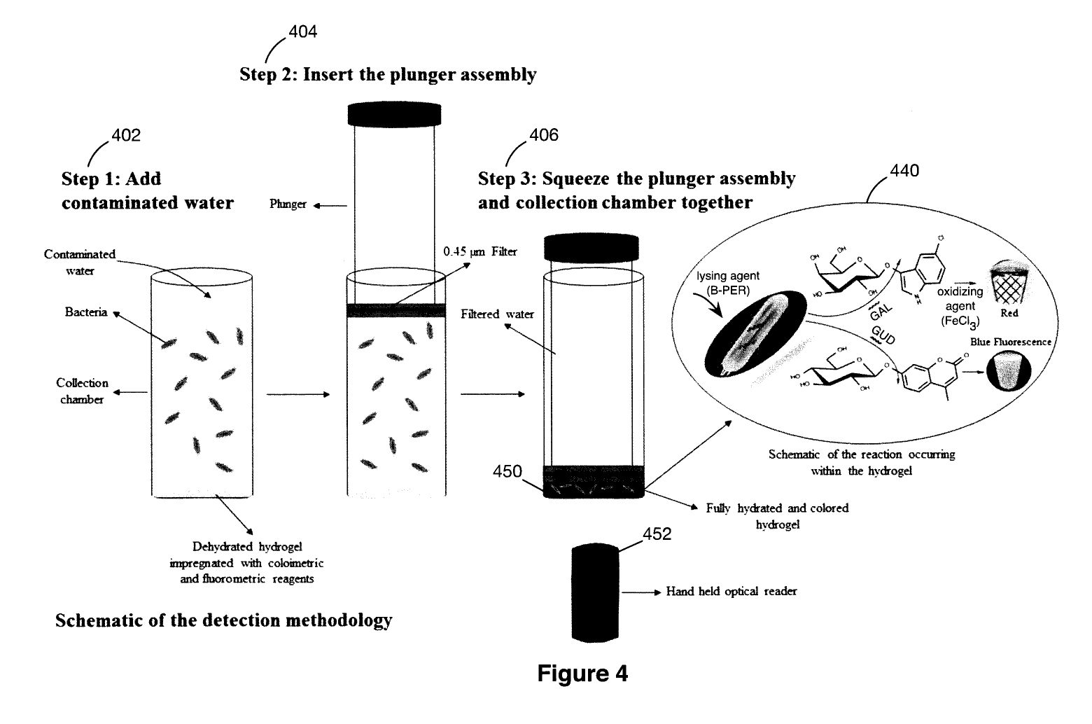

The working principle of this apparatus generally involves placing a fluid sample in the collection chamber 101 and then inserting the plunger 102 into the collection chamber 101. FIG. 2 shows the plunger 102 partially inserted into the collection chamber 101. The plunger 102 and the collection chamber 101 components are then squeezed together, thereby facilitating the movement of fluid and smaller particles into the plunger 102. FIG. 3 shows the plunger 102 more fully inserted into the collection chamber 101. After squeezing the plunger 102 and the collection chamber 101 together, larger particles within the fluid sample are trapped within the collection chamber 101 thereby concentrating particles of interest within the collection chamber 101. That is, in some embodiments, the plunger 102 and collection chamber 101 assembly, which may be referred to as a plunger-tube assembly, allows E. coli present in contaminated water to be concentrated in the head-space between the filter 110 (integrated with the plunger 102) and the bottom end 104 of the collection chamber 101, and provides for otherwise contaminant-free water to be collected inside the plunger 102.

The testing apparatus may be pre-treated with hydrogel impregnated with one or more substrates for marker enzymes of one or more pathogens. It will be appreciated that different substrates can be used depending on which pathogens are being tested for. The hydrogel may also comprise other ingredients to accelerate the reaction between the pathogens and the substrates.

In some embodiments, the bottom end 104 of the collection chamber may be pre-treated with the hydrogel. For illustrative simplicity, the hydrogel is not shown in FIGS. 1 to 3. It will be appreciated that a dried hydrogel may be used. The test liquid sample is then placed in the collection chamber 101. The plunger 102 is then placed into the collection chamber and moved into the collection chamber 101 so that some or all of the test water passes through the filter 110 in plunger tip 108. If the pathogen being tested for is present in the test liquid sample, the test liquid sample undergoes a change of colour. FIG. 3 depicts a test liquid sample 302 that has changed colour in this manner.

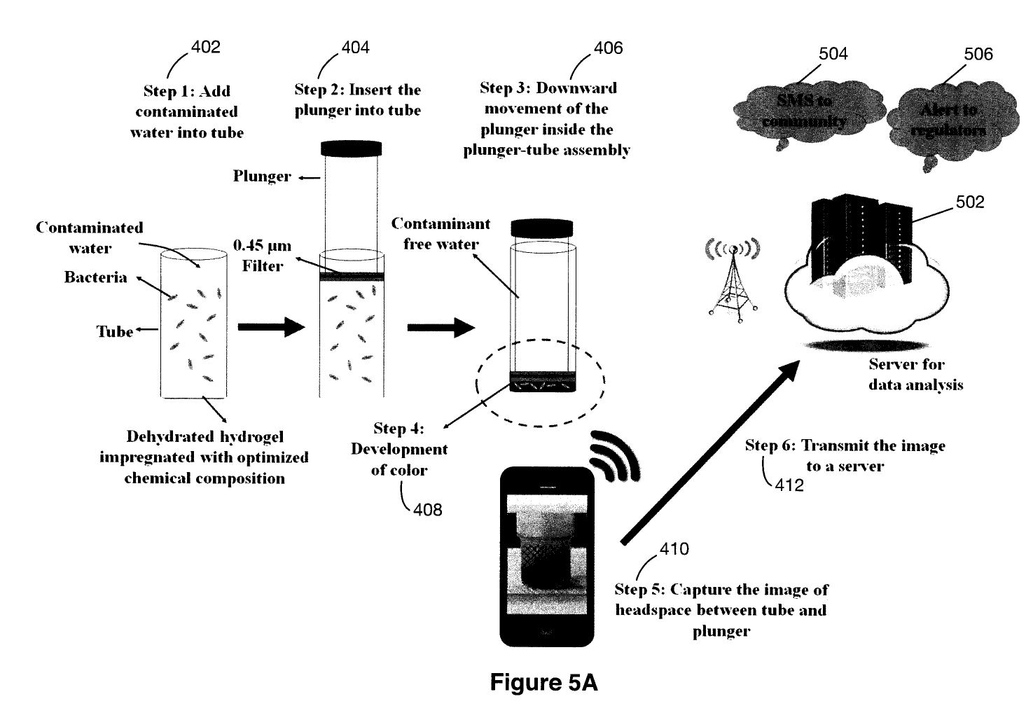

In some embodiments, the liquid being tested is water, and the pathogens are total coliform and E. coli. In such an embodiment, a dried hydrogel may be impregnated with Red-Gal and MUG. A schematic diagram depicting a testing method in such an embodiment is shown in FIG. 4. In the first step 402 of the testing method of this embodiment, a sample of water to be tested is collected and placed into the collection chamber 101. In the second step 404, the plunger 102 is inserted into the collection chamber 101 such that the plunger tip 108 is at the bottom of the plunger 102, and in the third step 406 the plunger 102 is depressed so that the plunger tip 108 is brought into contact, or proximity, with a dried hydrogel 450 at the bottom end of the collection chamber 104.

In some embodiments, if total coliform and/or E. coli are present in the water being tested, once the bacteria come in contact with the coated hydrogel, GUD and GAL are released in response to the available substrates within the hydrogel 450. In some embodiments, a lysis reagent such as a bacterial protein extraction reagent (B-PER) within the hydrogel 450 facilitates the release of the GUD and the GAL. A schematic of an example reaction occurring within the hydrogel 450 is shown within block 440 in FIG. 4. This reaction results in the breakdown of the substrates and quickly leads to the release of coloured and fluorescent molecules within the collection chamber. The increased surface area to volume ratio provided by the hydrogel and the reagent combination involved in promoting the reaction contribute to lessening the time for detection, i.e., the time that it takes for the release of coloured and fluorescent molecules within the collection chamber. If the water does not change colour, then the pathogens are not present in the water.

In some embodiments, the colour change is readily apparent without aid. In other embodiments, it will be appreciated that one or more lights may be required in order to verify the presence of a colour change, or lack thereof. In some embodiments, the colour change may be intended for quick qualitative measurements by an observer. In other embodiments, for quantitative results, the resultant colour can be measured using simple hand-held battery operated optical readers 452. An example of such a reader would include smartphones equipped with digital cameras, wherein the smartphone is optionally connected to communications networks. In some embodiments, the reader contains a custom mobile software application for analysis. In some embodiments, the reader contains calibrated look-up tables mapping particular colour intensities to known concentrations of E. coli.

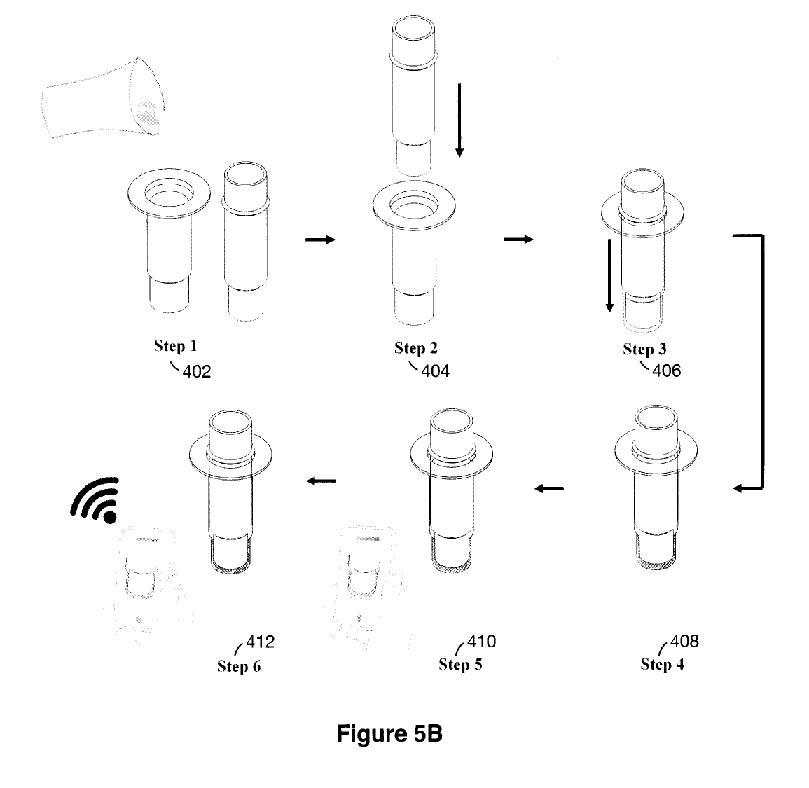

In some embodiments, quantitative results can be transmitted through communications networks, which may allow for results to be shared quickly, and over large distances, and for results to be monitored and exchanged. Results can be uploaded and transmitted by mobile devices such as, but not limited to, mobile phones, smartphones, tablets, and laptops. FIG. 5A is a schematic diagram depicting a variant of the detection methodology of FIG. 4, and FIG. 5B is an illustrative diagram depicting the steps involved. In the embodiment shown in FIGS. 5A and 5B, after a step 408 of waiting a period of time to allow for the development of colour, an image capture step 410 is performed by a smartphone to acquire an image of the space between the plunger and the collection chamber. In a subsequent step 412, the image is transmitted to a server 502 for data analysis. In some embodiments, server 502 is a cloud-based server. Results can be aggregated and monitored over time which can allow, for example, for public health officials to make decisions regarding water treatment in particular communities. For example, the data analysis may result in a Short Message Service (SMS) message 504 being sent to an affected community or an alert 506 being provided to regulators or healthcare professionals. In some embodiments, the messages and/or alerts may constitute a warning system that assists appropriate regulatory agencies, healthcare professionals, municipalities, and/or individuals to take the appropriate measures for treating tested water sources. In some embodiments, software on the server or the mobile device can access location-based services of the mobile device and thereby identify the test location and create a searchable water quality map for a community from multiple test results.

As described above, in some embodiments the pre-concentration of E. coli from 100 mL of water samples and colorimetric detection can be performed simultaneously within three simple steps 402, 404, 406. The time of detection is also greatly reduced compared to some other detection techniques due to the increased surface area to volume ratio provided by the hydrogel and also the reagent combination involved in promoting the reaction.

### Example 1

The following example demonstrates empirical results regarding chemical compositions for detection of contaminated samples, and is provided for the purposes of explanation, and not limitation, of the present invention.

Molecular biology grade agarose hydrogel and Tris/borate/EDTA (TBE) buffer were obtained from Biorad, Canada. Bacteria protein extraction reagent (B-PER), Difco Lauryl Tryptose Broth (LTB), Difco Nutrient Broth, BBL Brain Heart Infusion Broth, Difco Veal Infusion Broth and Bacto Yeast Extract were obtained from Fisher Scientific, Canada. 6-Chloro-3indolyl-β-D-galactopyranoside (also called Rose Gal, Salmon Gal or Red-Gal), 2-nitrophenyl-β-D-galactopyranoside (ONPG), 4-methylumbelliferyl-β-D-glucuronide trihydrate (4-MUG), 5-bromo-6-chloro-3-indolyl-β-D-glucopyranoside (Magenta Gluc), 4-methylumbelliferyl-β-D-galactopyranoside (MUGal), 5-bromo-4-chloro-3-indolyl-β-D-galactopyranoside (X-Gal), 4-methylumbelliferyl-β-D-glucuronide, dehydrate (MUG), and 5-bromo-6-chloro-3-indolyl-β-D-galactopyranoside (Magenta Gal) enzymatic substrates were procured from Sigma Aldrich, Canada. Nunc-Immuno MicroWell 96-well solid plates were obtained from Sigma Aldrich, Canada.

E. coli K-12 strains were obtained from New England Biolabs, Ipswich, Mass., USA. E. coli Castellani and Chalmers (American Type Culture Collection (ATCC) 11229), Enterococcus faecalis (E. faecalis) (Andrewes and Horder) Schleifer and Kilpper-Balz (ATCC 19433), Salmonella enterica subsp. enterica (S. enterica) (ex Kauffmann and Edwards) Le Minor and Popoff serovar Typhimurium (ATCC 14028) and Bacillus subtilis (B. substilis) (Ehrenberg) Cohn (ATCC 33712, MI112 strain) were purchased from Cedarlane, Burlington, ON, Canada. N,N-Dimethylformamide (DMF) and anhydrous ferric chloride (FeCl₃) were obtained from Sigma-Aldrich, Canada. Deionized (DI) water was used to prepare most of the solutions. Materials were sterilized whenever needed in an autoclave.

E. coli K-12 and E. coli ATCC 11229 strains were cultured in LTB medium at 37° C. in a microbiological incubator (Lab Companion SI-300 Benchtop Incubator and Shaker, GMI, Ramsey, Minn., USA) for 24 hours. Similarly, S. enterica and E. faecalis were grown in a Nutrient Broth and Brain Heart Infusion Broth, respectively. B. subtilis was grown in a medium consisting of Veal Infusion Broth and Yeast Extract in 5:1 ratio at 30° C. in an incubator for 24 h. Unless otherwise stated, serial dilutions were made in DI water to produce bacteria concentrations in the range of 400-4×10⁶ CFU mL⁻¹. These known bacteria concentration water samples were used to check the performance of newly formulated chemicals as described below.

The combination of chemicals used for the detection of E. coli consists of three key ingredients, henceforth referred to, as reagents X, Y, and Z. Chemical reagent X was prepared by dissolving a mixture of LTB in DI water. Chemical reagent Y was B-PER. Chemical reagent Z was prepared by dissolving an enzymatic substrate (30 mg) in 1 mL of a 1:1 mixture of DMF and DI water. All the chemical reagents were maintained at pH 7.

A method of detection was tested in the 96-well plates. The first step of the test method involved concentrating the bacteria samples in small volumes, by filtering 100 mL contaminated water samples with a syringe filter or a steri-cup filter. 100 μL of the pre-concentrated water samples containing bacteria was added to the wells of 96-well plates. A volume of 100 μL of reagent X, 25 μL of reagent Y, and 50 μL of reagent Z was then added sequentially to the wells. The plates were then incubated for one hour at 37° C. The appearance of color/fluorescence in the wells containing E. coli was observed. This test was conducted with different combinations of enzymatic substrates with different kinds of bacteria, metals and ions to find an optimized chemical composition for rapid and specific detection of E. coli.

More specifically, detailed studies were conducted to test and select an appropriate combination of reagents to detect E. coli in contaminated water samples. The chemical composition used in this work for detecting E. coli contains three components: (1) growth media to provide nutrients for the growth of E. coli; (2) a lysing agent to extract the enzymes from E. coli; and (3) enzymatic substrate to target specific enzymes (GAL or GUD) of E. coli. We used LTB as the growth media to provide nutrients to E. coli. We used lysing detergent (B-PER) to extract the enzymes associated with the E. coli bacteria. The lysing method employed here can extract the enzymes from E. coli within seconds and at the same time enhance the interaction between the enzymes and the chemicals to immediately release the colored or fluorescent chemicals for visualization and quantification. We selected several enzymatic substrates for the enzymes GAL and GUD thereby enabling detection of E. coli. Enzymatic substrates with β-D-galactopyranoside were used to target the GAL enzyme produced by E. coli in order to yield some color or fluorescent derivatives. Similarly, enzymatic substrates with β-D-glucuronic acid were used to target the GUD enzyme produced by E. coli in order to yield some color or fluorescent derivatives.

Initial experiments were conducted in the 96-well plates using several enzymatic substrates in the presence of LTB and B-PER. Table 1 below shows the list of enzymatic substrates used.

TABLE 1
List of eight different enzymatic substrates tested

| Enzymatic | |
|---|---|
| Substrate | |
| Label | Enzymatic Substrate |
| A | 6-chloro-3-indolyl-β-Dgalactopyranoside (Red-Gal) |
| B | 2-nitrophenyl-β-Dgalactopyranoside (ONPG) |
| C | 4-methylumbelliferyl-β-Dglucuronide, trihydrate (4-MUG) |
| D | 5-Bromo-6-chloro-3-indolyl-β-Dglucopyranoside |
| | (Magenta Gluc) |
| E | 4-methylumbelliferyl-β-Dgalactopyranoside (MUGal) |
| F | 5-bromo-4-chloro-3-indolyl-β-Dgalactopyranoside (X-Gal) |
| G | 5-bromo-6-chloro-3-indolyl-β-Dgalactopyranoside |
| | (Magenta Gal) |
| H | 4-methylumbelliferyl-β-Dglucuronide, dehydrate (MUG) |

Enzymatic substrates # A, B, F and G are chromogenic compounds, which produce color when they react with the GAL enzyme whereas enzymatic substrate # E is a fluorogenic compound, which produces blue fluorescence when it reacts with the GAL enzyme. The enzymatic substrate # A (Red-Gal) is an off-white to pale pink solid, which develops pinkish red to dark red color producing a dimerized 6-chloro3-indolyl compound when it interacts with the GAL enzyme. Enzymatic substrate # B (ONPG) is a white to off-white solid, which can be hydrolyzed by the GAL enzyme to produce a yellowish ortho-nitrophenol compound. Enzymatic substrate # F (X-Gal) is a white to off-white solid, which interacts with the GAL enzyme and produces a blue compound (5,5′-dibromo4,4′-dichloro indigo). The GAL enzyme produced by E. coli hydrolyses the white/off-white enzymatic substrate # G (Magenta Gal) resulting in the release of a magenta color producing compound. The enzymatic substrate # E (MUGal) is a white solid, which can be hydrolyzed by the β-D-galactosidase enzyme to release the blue fluorescence producing (4-MU) compound. Enzymatic substrates # C (4-MUG) and H (MUG) are fluorogenic compounds which produce a blue fluorescent molecule when they interact with the GUD (β-D-glucuronidase) enzyme. Enzymatic substrate # D (Magenta Gluc) is a colorimetric compound which reacts with the GUD enzyme to release a magenta color producing compound.

E. coli K-12 and ATCC 11229 were used as model E. coli bacteria for testing. In order to test the interference from other species of bacteria commonly found in water samples, experiments were conducted with different water samples containing several kinds of bacteria strains, metals and ions. Table 2 below provides the list of water samples used. E. faecalis, S. enterica, and B. subtilis were used as interference bacteria whereas cadmium, lead, sodium fluoride and sodium chloride were used as metallic and/or ionic contamination interferences. In total 21 different water samples were used to optimize the chemical composition.

TABLE 2
List of 21 different water samples tested

| Sample | |
|---|---|
| No. | Contents in Water Samples |
| 1 | E. coli K-12 (grown in LTB) |
| 2 | E. coli K-12 (grown in Nutrient Broth) |
| 3 | E. coli ATCC 11229 (cultured in LTB) |
| 4 | E. coli ATCC 11229 (cultured in Nutrient Broth) |
| 5 | E. faecalis |
| 6 | S. enterica |
| 7 | B. subtilis |
| 8 | B. subtilis |
| 9 | DI water without bacteria, metals, and ions |
| 10 | E. coli K-12, ATCC 11229, E. faecalis, S. enterica, |
| | and B. subtilis |
| 11 | E. coli K-12, E. faecalis, S. enterica, and B. subtilis |
| 12 | E. coli ATCC 11229, E. faecalis, S. enterica, and B. subtilis |
| 13 | E. coli K-12, ATCC 11229, and cadmium |
| 14 | E. coli K-12, ATCC 11229, and lead |
| 15 | E. coli K-12, ATCC 11229, and sodium fluoride |
| 16 | E. coli K-12, ATCC 11229, and sodium chloride |
| 17 | E. coli K-12, ATCC 11229, cadmium, lead, sodium |
| | fluoride and sodium chloride |
| 18 | E. coli K-12, ATCC 11229, E. faecalis, S. enterica, |
| | and B. subtilis, cadmium, |
| | lead, sodium fluoride, and sodium chloride |
| 19 | E. coli K-12 and ferric chloride |
| 20 | E. coli ATCC 11229 and ferric chloride |
| 21 | DI water with no bacteria, metals, and ions |

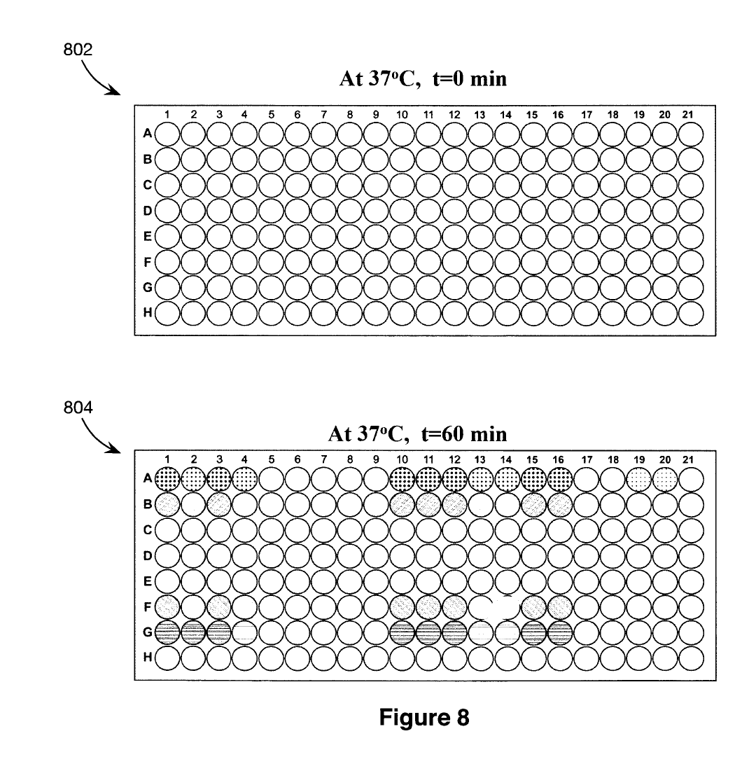

Enzymatic substrates # A, B, D, F, and G are colorimetric substrates. FIG. 8 shows the color development in several combinations of water samples containing E. coli and different interference bacteria, metals and/or ions after reacting with enzymatic substrates. In particular, FIG. 8 depicts a comparison of color development 802 at the start of the testing process, i.e., at t=0 min, and a comparison of color development 804 after t=60 min. Within each depicted time period, horizontal rows correspond to specific enzymatic substrates and vertical columns correspond to different water samples.

It was observed that the row with enzymatic substrate # D did not produce any color after reacting with water samples containing E. coli, which indicates that the model E. coli K-12 or ATCC 11229 does not have the GUD enzyme. Enzymatic substrates # A, B, F and G produced color after reacting with water samples containing E. coli (i.e., water samples #1-4). It was found that E. coli ATCC 11229 and E. coli K-12 grown in LTB released more intense colour compared to E. coli ATCC 11229 and E. coli K-12 grown in nutrient broth.

Enzymatic substrates # A, B, F and G produced color after reacting with water samples containing E. coli (i.e., water samples #1-4 and 10-16), whereas no notable color production was observed for water samples containing bacteria other than E. coli only (i.e., water samples #5-8). It was clearly indicated that the interference bacteria have no effect on the detection of E. coli with these enzymatic substrates (A, B, F and G). Water samples #10, 11 and 12 contain E. coli bacteria and the other interference bacteria including E. faecalis, S. enterica, and B. subtilis. It was observed that these water samples produce color with the enzymatic substrates (A, B, F and G), although they contain a mixture of interference bacteria.

Enzymatic substrates # A, B, F and G produced color after reacting with water samples containing E. coli (i.e., water samples #1-4 and 10-16). The metallic ions cadmium and lead produced less color intensity after reacting with water samples containing E. coli whereas the other samples produced more color intensity. Water samples #13, 14, 15 and 16 contained E. coli along with cadmium, lead, sodium fluoride and sodium chloride, respectively. However, these metallic and ionic solutions did not affect the production of color when they interacted with enzymatic substrates # A, B and G. Enzymatic substrate # F does not produce any color when it reacted with water samples containing E. coli along with cadmium and lead. Water samples #17 and 18 did not show any color though they had E. coli bacteria along with all the interference bacteria, metals and ions. The reason would be the number of E. coli bacteria is very low compared to other interfering bacteria, metals and ions. Hence, the color produced by E. coli bacteria was not detected by the naked eye.^{23,28}

Water samples #19 and 20 contained E. coli along with ferric chloride. Ferric chloride can be used as an oxidizing agent to intensify the color produced after reacting with the GAL enzyme. However, it was observed that ferric chloride containing water samples did not increase the color intensity compared to other samples. Ferric chloride is not a stable compound, it reacts with moisture in ambient air and becomes very acidic.^{29,30} Enzymatic substrates do not work properly in acidic solutions.^{31,32}

Water samples #9 and 21 contained DI water without bacteria, metals and ions. These samples also acted as negative controls. It was found that the enzymatic substrates did not produce any color for these samples.

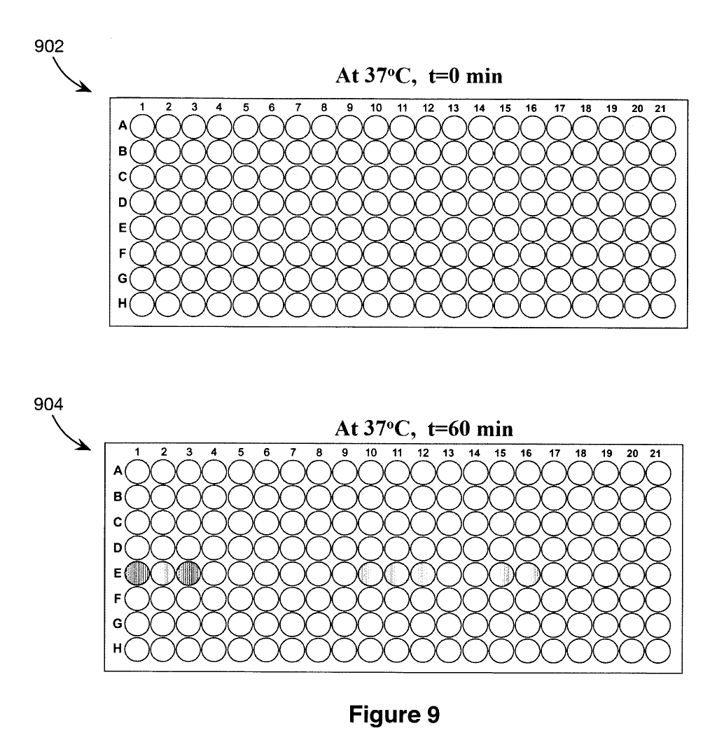

Enzymatic substrates # C, E and H are fluorogenic substrates. FIG. 9 shows the colour development in several combinations of water samples containing E. coli and different interference bacteria, metals and/or ions after reacting with enzymatic substrates. In particular, FIG. 9 depicts a comparison of fluorescence development 902 at the start of the testing process, i.e., at t=0 min, and a comparison of fluorescence development 904 after t=60 min. Within each depicted time period, horizontal rows correspond to specific enzymatic substrates and vertical columns correspond to different water samples. The fluorescence emitted by the water samples was visualized under a simple hand-held ultraviolet (UV) lamp (Cole-Parmer 8 watt UV Lamp with Dual 365 nm Wavelength Light Tubes, Cole-Parmer, Montreal, QC, Canada).

It was observed that the enzymatic substrates with a glucuronic acid molecule (i.e., C and H) did not produce any fluorescence after reacting with water samples with and without E. coli bacteria. This indicates that the GUD enzyme is not present in E. coli. The row with enzymatic substrate # E produced blue fluorescence after reacting with water samples containing E. coli (i.e., water samples #1-3), whereas the substrate # E did not produce any fluorescence for water samples that did not contain E. coli even if they had other bacteria (i.e., water samples #5-8). Water samples #17 and 18 did not show any fluorescence though they contain E. coli. This may be because of the mixture of metallic and ionic compounds in these water samples. Water samples #9 and 21 were negative controls containing DI water without any bacteria, metals and ions. Enzymatic substrates # C, E and H did not produce any color in DI water samples, which showed that the enzymatic substrates were working fine.

From the empirical results demonstrated in FIGS. 8 and 9, it is indicated that the E. coli K-12 and ATCC 11229 being used contain a GAL enzyme only. Enzymatic substrates # A, B, E, F and G are good for detecting E. coli bacteria. Enzymatic substrate # E is a fluorogenic compound and it requires a UV lamp to identify the blue fluorescence produced by the GAL enzyme whereas the other substrates do not require a UV lamp. Enzymatic substrate # F is not able to produce color in the presence of cadmium and lead. Enzymatic substrate # B produces a yellowish color in the presence of E. coli which may pose a significant challenge for detection when the water samples contain some organic matter such as algae. Enzymatic substrates # A and G have good specificity in E. coli detection, however, the enzymatic substrate # G is expensive compared to enzymatic substrate # A. From these results, was clear that enzymatic substrate # A (Red-Gal) as a substrate has merits compared to other substrates as it has good specificity for detecting E. coli also it is relatively inexpensive. Hence, an optimized chemical composition for detecting E. coli was determined to be a composition containing Red-Gal, B-PER, and LTB. Table 3 below summarizes the effects of an optimized chemical composition containing Red-Gal, B-PER, and LTB on the 21 different water samples referred to above in Table 2.

TABLE 3
Effects of optimized chemical composition (Red-Gal, B-PER,
and LTB) on the 21 different water samples tested

| | | Effect of optimized chemical |
|---|---|---|
| Sample | | composition (Red-Gal, B- |
| No. | Contents in Water Samples | PER, and LTB) |
| 1 | E. coli K-12 (grown in LTB) | Colour produced |
| 2 | E. coli K-12 (grown in Nutrient Broth) | Colour produced |
| 3 | E. coli ATCC 11229 (cultured in LTB) | Colour produced |
| 4 | E. coli ATCC 11229 (cultured in Nutrient | Colour produced |
| | Broth) | |
| 5 | E. faecalis | No colour |
| 6 | S. enterica | No colour |
| 7 | B. subtilis | No colour |
| 8 | B. subtilis | No colour |
| 9 | DI water without bacteria, metals, and ions | No colour |
| 10 | E. coli K-12, ATCC 11229, E. faecalis, | Colour produced |
| | S. enterica, and B. subtilis | |
| 11 | E. coli K-12, E. faecalis, S. enterica, and | Colour produced |
| | B. subtilis | |
| 12 | E. coli ATCC 11229, E. faecalis, S. enterica, and | Colour produced |
| | B. subtilis | |
| 13 | E. coli K-12, ATCC 11229, and cadmium | Colour produced |
| 14 | E. coli K-12, ATCC 11229, and lead | Colour produced |
| 15 | E. coli K-12, ATCC 11229, and sodium | Colour produced |
| | fluoride | |
| 16 | E. coli K-12, ATCC 11229, and sodium | Colour produced |
| | chloride | |
| 17 | E. coli K-12, ATCC 11229, cadmium, lead, | No colour |
| | sodium fluoride and sodium chloride | |
| 18 | E. coli K-12, ATCC 11229, E. faecalis, | No colour |
| | S. enterica, and B. subtilis, cadmium, lead, | |
| | sodium fluoride, and sodium chloride | |
| 19 | E. coli K-12 and ferric chloride | Light colour produced |
| 20 | E. coli ATCC 11229 and ferric chloride | Light colour produced |
| 21 | DI water with no bacteria, metals, and ions | No colour |

### Example 2

The following specific example demonstrates one embodiment of the present invention, and is provided for the purposes of explanation, and not limitation, of the present invention.

Whatman 0.45 μm syringeless filters were obtained from VWR, Canada. E. coli K-12 strains were obtained from New England Biolabs, Canada. Molecular biology grade agarose and Tris/borate/EDTA (TBE) buffer were obtained from Biorad, Canada. All other chemical reagents were obtained from Sigma Aldrich, Canada.

4% w/v agarose gel solution was prepared in TBE buffer and 400 μl of this solution was added to the collection chamber of the syringeless filters. 100 μl of lauryl tryptone broth (LTB) with MUG, 20 μl of 3% FeCl₃ solution, 20 μl of bacterial protein extraction reagent (B-PER) and 25 μl of 4% Red-Gal were then added to the hydrogel solution and allowed to solidify. The collection chambers were then dried under vacuum for 24 hours to obtain a dried thin layer of gel impregnated with the substrates for GUD and GAL (Red-Gal and MUG) and other ingredients to accelerate the reaction process. In this embodiment, the other ingredients include the 20 μL of 3% FeCl₃ solution, and the 20 μL of bacterial protein extraction reagent (B-PER). In another embodiment, a hydrogel solution was prepared by dissolving 100 mg of agarose hydrogel in 10 mL of TBE buffer. The optimized chemical composition (LTB, B-PER and Red-Gal) referred to above determined through systematic testing using 96-well plates, was then added to 100 μL of hydrogel solution kept inside the tube of the plunger-tube assembly and the entire hydrogel matrix with impregnated chemical compounds was allowed to solidify. The tubes were then dried under vacuum for 24 hours to obtain a thin layer of dried hydrogel, which occupied the bottom portion of the plunger-tube assembly.

The plunger assembly with a 0.45 μm pore size filter was then inserted into the collection chamber and the two components were squeezed together thereby concentrating the larger bacterial particles within the collection chamber as described above and depicted in FIG. 3.

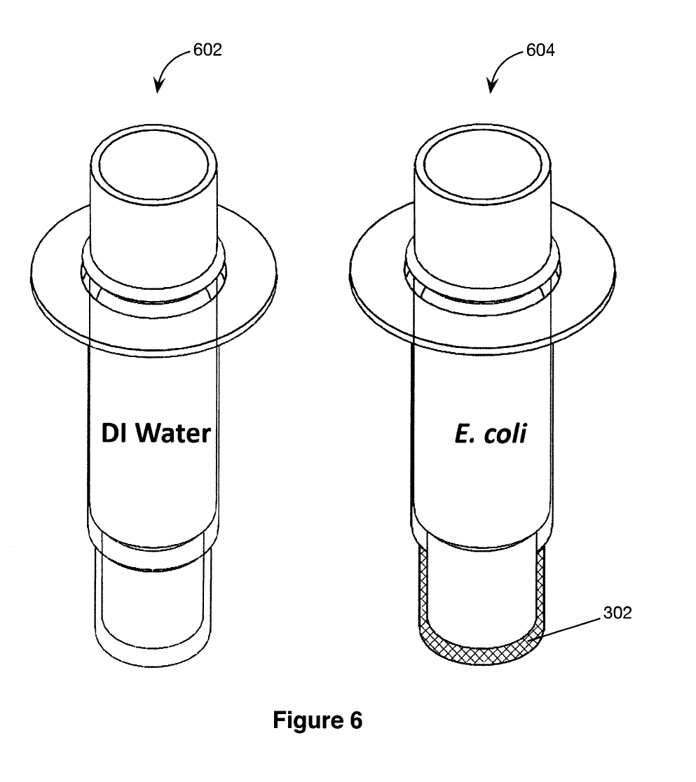

In the present example, once the bacteria came in contact with the coated hydrogel, GUD and GAL were released in response to the available substrates within the gel. This resulted in the breakdown of the substrates and lead to the release of coloured and fluorescent molecules within the collection chamber, and a visible colour was observed within the collection chamber indicating the presence of coliform bacteria. FIG. 6 shows two instances of the apparatus of the invention, the left hand apparatus 602 containing a control sample of DI water and the right hand apparatus 604 containing the test sample and showing development of colour 606 due to the enzymatic reaction of GAL with Red-Gal after 1 hour of incubation at 37° C. It is observed that there is a pinkish red color 606 appearing at the bottom of the tube to indicate the presence of E. coli whereas there is no color change in the control sample.

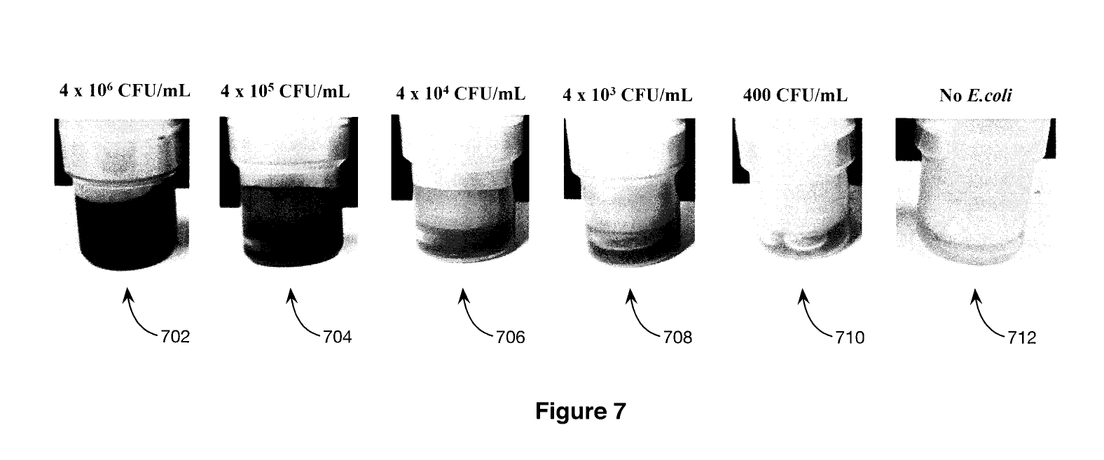

FIG. 7 shows development of color in samples 702, 704, 706, 708, 710 with decreasing concentrations of E. coli in an embodiment of the apparatus of the invention after 1 hour of incubation at 37° C. Sample 712 is a control sample of DI water. Note that the intensity of color change depends on the incubation time as well as the concentration of E. coli. The detection of E. coli was achieved within few minutes of incubation at 37° C. for higher concentrations of E. coli in the range of 4×10⁶ CFU mL⁻¹ to 4×10⁵ CFU mL⁻¹, whereas for concentrations in the range of 400 CFU mL⁻¹ to 4×10⁴ CFU mL⁻¹, the detection time was around 60 min. The intensity of the color produced by E. coli after reacting with the chemical composition depends on the number of E. coli bacteria. The color produced in the water samples 702, 704 (4×10⁶ CFU mL⁻¹ to 4×10⁵ CFU mL⁻¹) is able to be visualized in 5 min, whereas the color produced in the water samples 706, 708, 710 (4×10⁴ CFU mL⁻¹ to 400 CFU mL⁻¹) is able to be visualized after a longer time period. For concentrations of 4×10⁴ CFU mL⁻¹ to 400 CFU mL⁻¹, it may in some cases require 60 min or more to visualize the color by eye, and in some cases this may require under an hour. The number of E. coli bacteria will grow in LTB and produce more color over time. Note that the hydrogel matrix is used to store the chemical composition rather than playing a direct role in E. coli detection. The physical plunger-tube assembly plays a role in detection as it is able to screen out the target bacteria (E. coli in this case) from a given volume of water and concentrate it within the head-space between the plunger and the collection chamber.

The effects of different kinds of bacteria, metals, and ions on E. coli detection was also studied with the plunger-tube assembly. The results obtained with the plunger-tube assembly show analogous behavior as observed in the case of the 96-well plate method and described above.

As demonstrated, the plunger-tube assembly can pre-concentrate bacteria from water samples and also provide colorimetric results in a simple procedure which does not require a separate pre-concentration step or the sequential addition of chemical reagents. The detection method does not require any microbiology instruments and trained personnel, and thus may be of value for field deployment in limited resource communities.

Although the invention has been described with reference to certain specific embodiments, various modifications thereof will be apparent to those skilled in the art without departing from the spirit and scope of the invention.

### REFERENCES

1. Déportes, I.; Benoit-Guyod, J.-L.; Zmirou, D., Hazard to man and the environment posed by the use of urban waste compost: a review. Science of the Total Environment 1995, 172, (2), 197-222.
2. Girones, R., Tracking viruses that contaminate environments. Microbe-American Society for Microbiology 2006, 1, (1), 19-19.
3. Steele, M.; Odumeru, J., Irrigation water as source of foodborne pathogens on fruit and vegetables. Journal of Food Protection 2004, 67, (12), 2839-2849.
4. Olstadt, J.; Schauer, J.; Standridge, J.; Kluender, S., A comparison of ten USEPA approved total coliform E. coli tests. Journal of Water and Health 2007, 5, (2), 267-282.
5. Manafi, M., Chapter 12 Media for detection and enumeration of ‘total’ Enterobacteriaceae, coliforms and Escherichia coli from water and foods. Progress in Industrial Microbiology 2003, 37, (C), 167-193.
6. Olstadt, J.; Schauer, J. J.; Standridge, J.; Kluender, S., A comparison often USEPA approved total coliform/E-coli tests. Journal of Water and Health 2007, 5, (2), 267-282.
7. Grant, M. A., A new membrane filtration medium for simultaneous detection and enumeration of Escherichia coli and total coliforms. Applied and Environmental Microbiology 1997, 63, (9), 3526-3530.
8. Lifshitz, R.; Joshi, R., Comparison of a Novel ColiPlate Kit and the Standard Membrane Filter Technique for Enumerating Total Coliforms and Escherichia coli Bacteria in Water. Environmental Toxicology and Water Quality 1998, 13, (2), 157-164.
9. Noble, R. T.; Leecaster, M. K.; McGee, C. D.; Weisberg, S. B.; Ritter, K., Comparison of bacterial indicator analysis methods in stormwater-affected coastal waters. Water Research 2004, 38, (5), 1183-1188.
10. Noble, R. T.; Weisberg, S. B.; Leecaster, M. K.; McGee, C. D.; Ritter, K.; Walker, K. O.; Vainik, P. M., Comparison of beach bacterial water quality indicator measurement methods. Environmental Monitoring and Assessment 2003, 81, (1-3), 301-312.
11. Zhu, H.; Sikora, U.; Ozcan, A., Quantum dot enabled detection of Escherichia coli using a cell-phone. Analyst 2012, 137, (11), 2541-2544.
12. Comas-Riu, J.; Rius, N. r., Flow cytometry applications in the food industry. Journal of Industrial Microbiology & Biotechnology 2009, 36, (8), 999-1011.
13. Kong, R. Y. C.; Lee, S. K. Y.; Law, T. W. F.; Law, S. H. W.; Wu, R. S. S., Rapid detection of six types of bacterial pathogens in marine waters by multiplex PCR. Water Research 2002, 36, (11), 2802-2812.
14. Wéry, N.; Lhoutellier, C.; Ducray, F.; Delgenès, J.-P.; Godon, J.-J., Behaviour of pathogenic and indicator bacteria during urban wastewater treatment and sludge composting, as revealed by quantitative PCR. Water Research 2008, 42, (1), 53-62.
15. Heijnen, L.; Medema, G., Method for rapid detection of viable Escherichia coli in water using real-time NASBA. Water Research 2009, 43, (12), 3124-3132.
16. Afset, J. E.; Bruant, G.; Brousseau, R.; Harel, J. e.; Anderssen, E.; Bevanger, L.; Bergh, K. r., Identification of virulence genes linked with diarrhea due to atypical enteropathogenic Escherichia coli by DNA microarray analysis and PCR. Journal of Clinical Microbiology 2006, 44, (10), 3703-3711.
17. Bekal, S.; Brousseau, R.; Masson, L.; Prefontaine, G.; Fairbrother, J.; Harel, J. e., Rapid identification of Escherichia coli pathotypes by virulence gene detection with DNA microarrays. Journal of Clinical Microbiology 2003, 41, (5), 2113-2125.
18. Bruant, G.; Maynard, C.; Bekal, S.; Gaucher, I.; Masson, L.; Brousseau, R.; Harel, J. e., Development and validation of an oligonucleotide microarray for detection of multiple virulence and antimicrobial resistance genes in Escherichia coli. Applied and Environmental Microbiology 2006, 72, (5), 3780-3784.
19. Chen, S.; Zhao, S.; McDermott, P. F.; Schroeder, C. M.; White, D. G.; Meng, J., A DNA microarray for identification of virulence and antimicrobial resistance genes in Salmonella serovars and Escherichia coli. Molecular and Cellular Probes 2005, 19, (3), 195-201.
20. Jasson, V.; Jacxsens, L.; Luning, P.; Rajkovic, A.; Uyttendaele, M., Alternative microbial methods: an overview and selection criteria. Food Microbiology 2010, 27, (6), 710-730.
21. Bottari, B.; Ercolini, D.; Gatti, M.; Neviani, E., Application of FISH technology for microbiological analysis: current state and prospects. Applied Microbiology and Biotechnology 2006, 73, (3), 485-494.
22. Edberg, S. C.; Rice, E. W.; Karlin, R. J.; Allen, M. J., Escherichia coli: the best biological drinking water indicator for public health protection. Journal of Applied Microbiology 2000, 88, (S1), 106S-116S.
23. Manafi, M., New developments in chromogenic and fluorogenic culture media. International Journal of Food Microbiology 2000, 60, (2-3), 205-218.
24. Rompre, A.; Servais, P.; Baudart, J.; de-Roubin, M. R.; Laurent, P., Detection and enumeration of coliforms in drinking water: current methods and emerging approaches. Journal of Microbiological Methods 2002, 49, (1), 31-54.
25. Manafi, M., Fluorogenic and chromogenic enzyme substrates in culture media and identification tests. International Journal of Food Microbiology 1996, 31, (1), 45-58.
26. Manafi, M.; Kneifel, W.; Bascomb, S., Fluorogenic and chromogenic substrates used in bacterial diagnostics. Microbiological Reviews 1991, 55, (3), 335-348.
27. Gunda, N. S. K.; Naicker, S.; Shinde, S.; Kimbahune, S.; Shrivastava, S.; Mitra, S., Mobile Water Kit (MWK): A Smartphone Compatible Low-Cost Water Monitoring System for Rapid Detection of Total Coliform and E. coli. Analytical Methods 2014, 6, (16), 6236-6246.
28. Tryland, I.; Fiksdal, L., Enzyme Characteristics of β-D-Galactosidase- and β-D-Glucuronidase-Positive Bacteria and Their Interference in Rapid Methods for Detection of Waterborne Coliforms and Escherichia coli. Applied and Environmental Microbiology. 1998, 64, (3), 1018-1023.
29. Patnaik, P., Handbook of inorganic chemicals. McGraw-Hill, New York, 2003, vol. 1.
30. Audrieth, L. F., Inorganic Syntheses. McGraw-Hill Book Company, London, 1950.
31. Barman, T. E., Enzyme Handbook. Springer, 1969, vol. 2.
32. Kashket, E. R., Bioenergetics of lactic acid bacteria: cytoplasmic pH and osmotolerance. 1987, 46, (3), 233-244.

1. A hydrogel impregnated with one or more substrates for marker enzymes of one or more pathogens for use in a liquid testing kit, wherein when the hydrogel comes into contact with a liquid sample, if the one or more pathogens is present in the liquid sample, the hydrogel and liquid sample change colour due to a reaction process between the one or more substrates and the one or more pathogens.

2. The hydrogel of claim 1, wherein the one or more pathogens is total coliform.

3. The hydrogel of claim 1, wherein the one or more pathogens is E. coli.

4. The hydrogel of claim 1, wherein the one or more pathogens are total coliform and E. coli.

5. The hydrogel of claim 4, wherein the one or more substrates is 6-Chloro-3-indolyl-β-D-galactoside (Red-Gal).

6. The hydrogel of claim 3, wherein the one or more substrates is 4-Methylumbelliferyl β-D-glucoronide trihydrate (MUG).

7. The hydrogel of claim 4, wherein the one or more substrates are 6-Chloro-3-indolyl-β-D-galactoside (Red-Gal) and 4-Methylumbelliferyl β-D-glucoronide trihydrate (MUG).

8. The hydrogel of claim 3, wherein the one or more substrates is 5-bromo-6-chloro-3-indolyl-β-Dgalactopyranoside (Magenta Gal).

9. (canceled)

10. (canceled)

11. (canceled)

12. (canceled)

13. (canceled)

14. (canceled)

15. (canceled)

16. (canceled)

17. (canceled)

18. An apparatus for testing for the presence of one or more pathogens in a liquid sample, the apparatus comprising:
(a) a collection chamber;
(b) a plunger comprising at least one plunger wall and a plunger tip, wherein the at least one plunger wall is sized to fit in sliding contact with and move longitudinally within the collection chamber when the plunger is raised or lowered relative to the collection chamber;
(c) said plunger tip comprising a filter with pores, said pores sized to permit liquid to pass therethrough, but to not permit the one or more pathogens to pass therethrough; and
(d) a testing composition comprising one or more substrates for marker enzymes of one or more pathogens.

19. The apparatus of claim 18, wherein the one or more pathogens is at least one of total coliform and E. coli.

20. (canceled)

21. (canceled)

22. The apparatus of claim 19, wherein the one or more substrates is 6-Chloro-3-indolyl-β-D-galactoside (Red-Gal).

23. The apparatus of claim 19, wherein the one or more substrates is 4-Methylumbelliferyl β-D-glucoronide trihydrate (MUG).

24. The apparatus of claim 19, wherein the one or more substrates are 6-Chloro-3-indolyl-β-D-galactoside (Red-Gal) and 4-Methylumbelliferyl β-D-glucoronide trihydrate (MUG).

25. The apparatus of claim 19, wherein the one or more substrates is 5-bromo-6-chloro-3-indolyl-P-Dgalactopyranoside (Magenta Gal).

26. The apparatus of claim 19, wherein the testing composition is a hydrogel impregnated with one or more substrates for marker enzymes of one or more pathogens, wherein when the hydrogel comes into contact with the liquid sample, if the one or more pathogens is present in the liquid sample, the hydrogel and liquid change colour due to a reaction process between the one or more substrates and the one or more pathogens.

27. (canceled)

28. (canceled)

29. (canceled)

30. A method of testing for the presence of one or more pathogens in a liquid sample, the method comprising:
(a) collecting the liquid sample in a collection chamber;
(b) inserting into the collection chamber a plunger comprising at least one plunger wall and a plunger tip, wherein the at least one plunger wall is sized to fit in sliding contact with and move longitudinally within the collection chamber when the plunger is raised or lowered relative to the collection chamber, the plunger tip comprising a filter with pores, said pores sized to permit liquid to pass therethrough, but to not permit said one or more pathogens to pass therethrough;
(c) moving the plunger tip into the collection chamber such that a quantity of the one or more pathogens is retained on the filter as the liquid is passed therethrough which brings the quantity of one or more pathogens into contact with a testing composition comprising one or more substrates for marker enzymes of the one or more pathogens; and
(d) observing the testing composition and liquid for a colour change, said colour change signifying the presence of said one or more pathogens.

31. The method of claim 30, wherein the one or more pathogens is at least one of total coliform and E. coli.

32. (canceled)

33. (canceled)

34. The method of claim 31, wherein the one or more substrates is 6-Chloro-3-indolyl-β-D-galactoside (Red-Gal).

35. The method claim 31, wherein the one or more substrates is 4-Methylumbelliferyl β-D-glucoronide trihydrate (MUG).

36. The method of claim 31, wherein the one or more substrates are 6-Chloro-3-indolyl-β-D-galactoside (Red-Gal) and 4-Methylumbelliferyl β-D-glucoronide trihydrate (MUG).

37. The method of claim 31, wherein the one or more substrates is 5-bromo-6-chloro-3-indolyl-β-Dgalactopyranoside (Magenta Gal).

38. The method of claim 31, wherein the testing composition is a hydrogel impregnated with one or more substrates for marker enzymes of one or more pathogens, wherein when the hydrogel comes into contact with the liquid sample, if the one or more pathogens is present in the liquid sample, the hydrogel and liquid change colour due to a reaction process between the one or more substrates and the one or more pathogens.

39. (canceled)

40. (canceled)

41. (canceled)

42. (canceled)

43. (canceled)

44. (canceled)
